SEQUENCE LISTING (3 sequences)

SEQ ID NO: 1 - DNA, 464 bp, Artificial Sequence
  Bidirectional promoter for small RNA expression
agatctttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccggaagcttaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgagacc

SEQ ID NO: 2 - DNA, 198 bp, Artificial Sequence
  Bidirectional promoter for small RNA expression
ctcgagttcgttcgagttcgtcctttccacaagatatataaagccaagaaatcgaaatactttcaagttacggtaagcatatgatagtggtaccctgcaatatttgcatgtcgctatgtgttctgggaaatcaccataaacgtgaaatgtctttggatttgggaatcttataagttctgtatgagaccactcccatgg

SEQ ID NO: 3 - DNA, 5508 bp, Artificial Sequence
  Bidirectional promoter for small RNA expression
ttaactataacggtcctaaggtagcgatccatcatcataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgccggatgtggtaaaagtgacgtttttggtgtgcgccggtgtatacgggaagtgacaattttcgcgcggttttaggcggatgttgtagtaaatttgggcgtaaccaagtaatgtttggccattttcgcgggaaaactgaataagaggaagtgaaatctgaataattctgtgttactcatagcgcgtaatatttgtctagggccgcggggactttgaccgtttacctaggcccccttaaggccggccaattgaataaagccaaaataaaaaaaaaaaaaatgtgtgttggttttttgtggcaaaccacactttttttttaatttttatttttttttatttttctagctagacgcactagtgaaaaagacgcgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattctaattatctctctaacagccttgtatcgtatatgcaaatatgaaggaatcatgggaaataggccctcttcctgcccgaccttaagcttccggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaagatctgtttttgaattcgcggccgcaggtaagccagcccaggcctcgccctccagctcaaggcgggacaggtgccctagagtagcctgcatccagggacaggccccagccgggtgctgacacgtccacctccatctcttcctcaggtctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttaacttgtttattgcagcttataatggttacaaataaagcaatagcatcacaaatttcacaaataaagcatttttttcactgcattctagttgtggtttgtccaaactcatcaatgtatcttatcatgtctggatccgaattcaaaaactcgagttcgtcctttccacaagatatataaagccaagaaatcgaaatactttcaagttacggtaagcatatgatagtggtaccctgcaatatttgcatgtcgctatgtgttctgggaaatcaccataaacgtgaaatgtctttggatttgggaatcttataagttctgtatgagaccactctgtacactttttggcgcgcctatcgattcgaacggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcacttgatgcctccgtgtaagggggaatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgcgcacccgtggccaggacccaacgctgcccgagatgcgccgcgtgcggctgctggagatggcggacgcgatggatatgttctgccaaggttccattgttcattccacggacaaaaacagagaaaggaaacgacagaggccaaaaagcctcgctttcagcacctgtcgtttcctttcttttcagagggtattttaaataaaaacattaagttatgacgaagaagaacggaaacgccttaaaccggaaaattttcataaatagcgaaaacccgcgaggtcgccgccccgtaacctgtcggatcaccggaaaggacccgtaaagtgataatgattatcatctacatatcacaacgtgcgtggaggccatcaaaccacgtcaaataatcaattatgacgcaggtatcgtattaattgatctgcatcaacttaacgtaaaaacaacttcagacaatacaaatcagcgacactgaatacggggcaacctcatgtcgcattcacagttctccgcaagaattgattggctccaattcttggagtggtgaatccgttagcgaggtgccgccggcttccattcaggtcgaggtggcccggctccatgcaccgcgacgcaacgcggggaggcagacaaggtatagggcggcgcctacaatccatgccaacccgttccatgtgctcgccgaggcggcataaatcgccgtgacgatcagcggtccagtgatcgaagttaggctggtaagagccgcgagcgatccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccgccatgccggcgataatggcctgcttctcgccgaaacgtttggtggcgggaccagtgacgaaggcttgagcgagggcgtgcaagattccgaataccgcaagcgacaggccgatcatcgtcgcgctccagcgaaagcggtcctcgccgaaaatgacccagagcgctgccggcacctgtcctacgagttgcatgataaagaagacagtcataagtgcggcgacgatagtcatgccccgcgcccaccggaaggagctgactgggttgaaggctctcaagggcatcggtcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagagtatccacaggacgggtgtggtcgccatgatcgcgtagtcgatagtggctccaagtagcgaagcgagcaggactgggcggcggccaaagcggtcggacagtgctccgagaacgggtgcgcatagaaattgcatcaacgcatatagcgctagcagcacgccatagtgactggcgatgctgtcggaatggacgatatcccgcaagaggcccggcagtaccggcataaccaagcctatgcctacagcatccagggtgacggtgccgaggatgacgatgagcgcattgttagatttcatacacggtgcctgactgcgttagcaatttaactgtgataaactaccgcattatagcttatcgaagataagctgtcaaacatgagaa

(19) United States
(12) Patent Application Publication
(10) Pub. No.: US 2006/0171924 A1
(43) Pub. Date: Aug. 3, 2006

(54) Bidirectional promoters for small RNA expression

(76) Inventors: Ke Luo, Worcester, MA (US); Ling Du, Framingham, MA (US); G. Peter Frick, Framingham, MA (US)

Correspondence Address: Gary F. Wang, Esq., Suite 302, 201 South Lake Avenue, Pasadena, CA, 91101, US

(21) Appl. No.: 10/957,407

(22) Filed: Oct. 2, 2004

Related U.S. Application Data
(60) Provisional application No. 60/508,821, filed on Oct. 3, 2003.

(51) Int. Cl. A61K 48/00; C12N 15/861

(52) U.S. Cl. 424/93.2; 435/456

(57) ABSTRACT

The invention provides bidirectional promoters for expressing two or more short RNA sequences from a single promoter. A particular embodiment of the bidirectional promoters of the invention include: 1) a Pol III promoter that contains a TATA box, a PSE and a DSE; and 2) a Pol III promoter that includes a PSE and a TATA box fused to the 5′ end of said DSE in reverse orientation. Vector embodiments are also disclosed comprising the novel bidirectional promoters of the invention, as well as methods of making and using these promoters.

### RELATED APPLICATIONS

The present application is related to and claims priority from the provisional application filed on Oct. 3, 2003, Ser. No. 60/508,821.

### BACKGROUND OF THE INVENTION

1. Field of the Invention

The present invention relates generally to RNA polymerase promoters and more specifically it relates to bidirectional RNA polymerase promoters that direct the expression of short RNA sequences, such as those used to silence gene expression.

2. Description of the Prior Art

Low efficiency of RNA delivery into cells appears to be the major problem in RNA mediated gene silencing. Another main limitation with the current vector based RNAi delivery approaches using U6 and H1 Pol III promoters is that they can direct the expression of only one short RNA sequence, and thus can only be used silence only one gene segment of one gene. Instances remain where it is desirable to silence two or more gene segments in one or more genes at a time. Targeting two gene segments of a particular gene of interest would be expected to increase the efficiency of targeted gene silencing. Further, effective gene silencing to overcome certain disease states may require that two or more genes be targeted for gene silencing. A general example of such a disease state is found in certain cancers where more than one gene is generally involved in a particular disease state phenotype. From an efficiency standpoint, as well as from the standpoint of treating certain disease states, it would be advantageous to express more than one short RNA sequence directed to silencing one or more genes of interest. However, the ability to direct the expression of two or more short RNA sequences is unavailable for gene silencing is currently unavailable.

The efficiency of the RNA mediated gene silencing generally would be expected to increase with the number of short RNA sequences targeted to the one or more genes of interest. However, if one wants to express more than one short RNA (shRNA) sequence by assembling more than one Pol III promoter in tandem, the repeated sequences cause vector instability and eventually result in termination of expression (De Wilde, C. et al. 2000, Plant Mol. Biol. 43:347-359), thus efficiency of the RNA mediated gene silencing is greatly diminished. While Pol III promoters such as U6 and H1 may be suitable for the expression of single shRNA sequences, they are not as suitable for expressing two or more shRNA sequences.

Thus, there is a need in the art of targeted RNA mediated gene silencing to produce two or more shRNA sequences from a single vector without the negative effect of vector instability and early termination of the targeted expression. If such a vector construct could be created, the efficiency of targeted RNA gene silencing using shRNA sequences could be increased, as well as increasing the number of genes segments within a single gene that can be targeted at the same time. Also such technology would permit multi-gene targeting. If these desired features of RNA mediated gene silencing could be achieved, the resultant vectors constructs would facilitate the use of RNA gene silencing in many clinical applications in medicine and basic research.

### SUMMARY OF THE INVENTION

Embodiments of the present invention provide promoters, vector constructs and related methods that achieve the desired effect of efficient and/or multi-gene targeted RNA silencing. In view of the disadvantages inherent in the known types of Pol III promoters of the prior art, the embodiments of the present invention provide novel bidirectional Pol III promoters for directing the expression of short RNA sequences in eukaryotic cells where each bidirectional Pol III promoter can direct the expression of two short RNA sequences. Related embodiments include vector constructs that comprise one or more bidirectional promoters of the present invention. These vector constructs comprise any nucleic acid that includes the bidirectional promoters of the present invention, such as plasmid or viral vector constructs.

When one or more bidirectional promoters are employed the efficiency and effectiveness delivering of multiple, distinct short RNA sequences for targeted gene silencing is improved; Generally, the bidirectional promoters of the invention direct the expression of different short RNA sequences. However, multiple copies of the same short RNA sequences can also be expressed via direction of the bidirectional promoters of the invention. Further, the bidirectional Pol III promoters of the present invention retain the advantages of conventional Pol III promoters with improved efficiency, as well as the capability of multi-gene targeting. Thus, the bi-directional promoters of the invention are not anticipated, rendered obvious, suggested, or even implied by any of the prior art Pol III promoters, either alone or in any combination.

To attain the advantages of expressing two or more short RNAs for targeted gene silencing, the present invention generally provides a bidirectional RNA polymerase III promoter. Embodiments of the bidirectional promoters of the present invention generally comprise certain control elements that provide for transcription of short RNA sequences in one direction, and certain control elements added in reverse orientation that provide for transcription of short RNA sequences in the opposite direction. Other embodiments of the invention include vectors comprising one or more bidirectional promoters. The vector embodiments of the invention generally include plasmids, viral constructs, or the like, that include the novel bidirectional promoters disclosed herein. The vector embodiments of the invention can be furthered packaged to facilitate delivery of the vector into a cell. An example of such an embodiment would be an adenovirus construct containing a vector embodiment of the invention that includes the necessary viral packaging to facilitate delivery into a cell. However, other delivery systems, i.e., other than viral delivery systems, are included in specific embodiments of the present invention. These delivery systems include liposomes or the like. These delivery systems could be used to deliver non-viral DNA vector into a cell.

In particular, embodiments of the bidirectional promoters of the invention comprises 1) a Pol III promoter, which includes a TATA box, a Proximal Sequence Element (PSE), and a Distal Sequence Element (DSE), and 2) a second Pol III promoter, including a PSE and TATA box fused to the 5′ terminus of said DSE in reverse orientation. Generally, the embodiments of the invention comprising bidirectional Pol III promoters utilize conventional, unidirectional Pol III promoters, the U6 and H1 promoters, as a starting point, and then rearrange, reorient or alter the promoters to render them bidirectional.

Particular embodiments of the invention include plasmids comprising elements that facilitate propagation in a bacterial host and subsequent short RNA expression in eukaryotic cells from one or more bidirectional Pol III promoters of the invention. Other embodiments of the invention include viral delivery of vector constructs that include one or more bidirectional promoter of the invention, where the vector constructs are capable of expressing two or more short RNAs for gene silencing following entry of the viral delivery system into a cell of interest. A particular embodiment of the invention that includes a viral delivery system comprises adenovirus including elements that facilitate the expression of short RNAs for gene silencing from one or more bidirectional RNA polymerase III promoters of the invention. The adenovirus embodiment is used as example to demonstrate the efficiency of the bidirectional promoter embodiments of the invention in silencing endogenous genes. Further the adenovirus embodiment of the invention illustrates that such viral delivery systems allow for highly efficient gene transfer into host cells. The adenovirus embodiment further provides an efficient method for directing expression of two or more different short RNA sequences comprising a vector that includes one or more bidirectional RNA polymerase III promoters to direct transcription of the short RNA sequences in a host cell of interest.

Other embodiments of the invention include methods of silencing the expression of two or more genes comprising the expression of two or more short RNA sequences under the direction of one or more bidirectional RNA polymerase III promoters. These embodiments of the invention includes methods for increasing the number of short RNA sequences that are expressed under the direction of elements of a single vector comprising the inclusion of one or more bidirectional RNA polymerase III promoters in said vector.

Still other embodiments of the invention include methods for treating disease in a subject, preferably a human, comprising administration of a vector of the invention that includes one or more bidirectional RNA polymerase III promoters. Particular examples includes methods for treating an infectious disease in a subject, such as HIV/AIDS, herpes, hepatitis or the like. These methods generally comprise targeting two or more regions of a particular viral genome to obtain efficient suppression of the expression of the virus in the subject. Thus, these methods targets two or more regions of the viral genome simultaneously or near simultaneously by administering an embodiment of a vector of the invention. In these methods of the invention, the particular vector embodiment would include one or more bidirectional RNA polymerase III promoters that are capable of directing the expression of two or more viral-related short RNA sequences to yield efficient suppression of the expression of the viral-related genes. Very efficient suppression of virally related genes would reduce the number of virus in the subject. Viral-related RNA sequences are those RNA sequences constructed to be complementary to one or more viral sequences.

Also included in the present invention are methods for minimizing the size of control elements that direct transcription of short RNA sequences comprising the inclusion of one or more bidirectional RNA polymerase III promoters to said control elements.

Further embodiments of the invention include kits comprising the bidirectional Pol III promoters of the invention and instructions for use. The kit embodiments may include vectors that comprise the bidirectional Pol III promoters, such as plasmid or viral vector, or may include the bidirectional Pol III promoters in such a manner so as to facilitate the construction of a vector. In either case, a kit embodiment of the invention provides the means to express two or more short RNA sequences directed by one or more bidirectional RNA polymerase III promoters of the invention.

There has thus been outlined, rather broadly, important features of the invention in order that the detailed description thereof may be better understood, and in order that the present contribution to the art may be better appreciated. There are additional features of the invention that will be described.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

One object of the present invention is to provide bidirectional Pol III promoters that will overcome the shortcomings of the prior art devices.

One object of the present invention is to provide a bidirectional Pol III promoter that directs the expression of two different short RNA sequences.

Another object is to provide a means to express multiple short RNA sequences under the control of regulatory elements that reside within a single vector by inserting one or more bidirectional Pol III promoters into said vector.

Another object is to provide bidirectional Pol III promoters that can silence gene expression more efficiently than conventional Pol III promoters by expressing multiple short RNA sequences that target multiple regions of the gene to be silenced.

Another object is to reduce the uncertainty that is encountered when individual short RNA sequences are used in an attempt to silence gene expression by providing vectors that can express multiple short RNA sequences. It can be appreciated that if an individual sequence may fail to achieve the desired result, the use of multiple sequences can be expected to reduce the failure rate.

Another object is to silence the expression of two or more genes in a coordinated fashion by expressing multiple short RNA sequences under the direction of bidirectional Pol III promoters in a single vector.

Another object is to silence the expression of two or more genes of a signaling transduction pathway in a tandem or a parallel fashion by expressing multiple short RNA sequences under the direction of bidirectional promoters in a single vector.

Another object is to minimize the size of bidirectional Pol III promoters so that they can be inserted into any vector, especially into those having size limitations such as adeno-associated virus and lentivirus vectors.

Another object is to provide a hybrid bidirectional Pol III promoter for maximal transcription activity with little or no repeated sequences in the promoter.

Other objects and advantages of the present invention will become obvious to the reader, and it is intended that these objects and advantages are within the scope of the present invention. To the accomplishment of the above and related objects, this invention may be embodied in the form illustrated in the accompanying drawings, attention being called to the fact, however, that the drawings are illustrative only, and that changes may be made in the specific construction illustrated.

### BRIEF DESCRIPTION OF THE DRAWINGS

Various other objects, features and attendant advantages of the present invention will become fully appreciated as the same becomes better understood when considered in conjunction with the accompanying drawings, in which like reference characters designate the same or similar parts throughout the several views, and wherein:

FIG. 1 is a diagram of a bidirectional Pol III promoter of the present invention.

FIG. 2 is the nucleotide sequence of the human BiU6 promoter.

FIG. 3 is the nucleotide sequence of the human BiH1 promoter.

FIG. 4 illustrates features of a plasmid, pBiU6, constructed to show proof of principle of the effectiveness of BiU6 promoter.

FIG. 5 compares silencing of a reporter gene by short RNA sequences expressed under the direction of either the forward or the reverse oriented basic promoter of the BiU6 promoter using quantitative enzymatic assay.

FIG. 6 demonstrates silencing of a reporter gene by short RNA sequences expressed under the direction of either the forward or the reverse oriented basic promoter of the BiU6 promoter in x-gal stained cells.

FIG. 7 illustrates features of a plasmid, pBiH1 constructed to show proof of principle of the effectiveness of the BiH1 promoter.

FIG. 8 illustrates features of a plasmid, pQuiet4, constructed to show proof of principle of the effect of using four positions for shRNA expression from BiU6 and BiH1 promoters.

FIG. 9 compares silencing of a reporter gene by short RNA sequences expressed under the direction of either the forward or the reverse oriented promoters of the BiU6 and the BiH1 promoters, i.e. at each of the four positions individually.

FIG. 10 demonstrates silencing of three reporter genes simultaneously by short RNA sequences expressed from both direction of forward and reverse U6 promoter and the forward H1 promoter.

FIG. 11 Illustrates features of a plasmid, pQuiet p38-3, that contains three shRNA sequences to target mouse p38 kinase isoforms α, β and γ.

FIG. 12 demonstrates silencing of three endogenous genes (three isoforms of mouse p38 kinase) simultaneously in cultured cells by an adenoviral vector that expresses three p38 shRNAs.

### DETAILED DESCRIPTION

Embodiments of the present invention provide promoters, vector constructs and related methods that achieve the desired effect of efficient and/or multi-gene targeted RNA silencing. In view of the disadvantages inherent in the known types of Pol III promoters of the prior art, the embodiments of the present invention provide novel bidirectional Pol III promoters for directing the expression of short RNA sequences in eukaryotic cells where each bidirectional Pol III promoter can direct the expression of two short RNA sequences. Further, although specific embodiments of the bidirectional promoters of the invention depict RNA Pol III promoters, in its broadest aspect the invention encompasses the utilization of other RNA polymerase promoters to create bidirectional promoters. Related embodiments include vector constructs that comprise one or more bidirectional promoters of the present invention. These vector constructs comprise any nucleic acid that includes the bidirectional promoters of the present invention, such as plasmid or viral vector constructs. To attain the advantages of expressing two or more short RNAs for targeted gene silencing, the present invention generally provides a bidirectional RNA polymerase III promoter.

Embodiments of the bidirectional promoters of the present invention generally comprise certain control elements that provide for transcription of short RNA sequences in one direction, and certain control elements added in reverse orientation that provide for transcription of short RNA sequences in the opposite direction. Other embodiments of the invention include vectors comprising one or more bidirectional promoters. The vector embodiments of the invention generally include plasmids, viral constructs, or the like, that include the novel bidirectional promoters disclosed herein. The vector embodiments of the invention can be furthered packaged to facilitate delivery of the vector into a cell. An example of such an embodiment would be an adenovirus construct containing a vector embodiment of the invention that includes the necessary viral packaging to facilitate delivery into a cell. However, other delivery systems, i.e., other than viral delivery systems, are included in specific embodiments of the present invention. These delivery systems include liposomes or the like. These delivery systems could be used to deliver non-viral DNA into a cell.

The bidirectional Pol III promoters described in the present invention substantially depart from conventional concepts and designs of the prior art, and in so doing provide vectors vehicles primarily developed for the purpose of expressing two short RNA sequences from each bidirectional Pol III promoter. It can be appreciated that RNA polymerase III dependent promoters (referred to hereafter as Pol III) have been in use for years, and that the properties of the U6 and H1 promoters have made them popular choices for the expression of short RNA sequences that reduce, or “silence” gene expression in eukaryotic cells. Short sequences of RNA that are expressed for this purpose usually have a hairpin configuration to create a double stranded region and are referred to as shRNA (short hairpin RNA) sequences or expressed RNAi (RNA interference). The term “short RNA sequences” will be used in this document, and is meant to include shRNA and expressed RNAi sequences but can also include other RNA sequences generally up to approximately 300 nucleotides in length such as antisense RNA and ribozyme sequences

While Pol III promoters such as U6 and H1 may be suitable for the expression of single short RNA sequences, they are not as suitable for expressing two or more short RNA sequences, which now can be achieved using bidirectional Pol III promoters of the present invention. In this respect, the bidirectional Pol III promoters disclosed in the present invention substantially depart from conventional concepts and designs of the prior art, and in so doing provide vector vehicles primarily developed for the purpose of expressing two short RNA sequences from each bidirectional Pol III promoter.

In particular, embodiments of the bidirectional promoters of the invention comprise 1) a Pol III promoter, which includes a TATA box, a Proximal Sequence Element (PSE), and a Distal Sequence Element (DSE), and 2) a second Pol III promoter, including a PSE and TATA box fused to the 5′ terminus of said DSE in reverse orientation. Generally, the embodiments of the invention comprising bidirectional Pol III promoters utilize conventional, unidirectional Pol III promoters, the U6 and H1 promoters, as a starting point, and then rearrange, reorient or alter the promoters to render them bidirectional.

It has been estimated that as many as 40,000 RNA transcripts per cell can be made by promoters such as U6 and H1 under optimized experimental conditions. This level of gene expression is sufficient to reduce gene expression efficiently through expression of shRNA or RNAi (Tuschl, T. 2002, Nat. Biotechnol., 48:446-448), providing that an effective shRNA sequence is expressed. However, selection of effective shRNA sequences is a haphazard process at the present time, although there are a few rules that provide some guidance. Even proprietary, sophisticated computer algorithms cannot guarantee that a selected shRNA sequences will work well. It appears that the effectiveness of selected sequences varies widely and that a degree of uncertainty concerning the effectiveness of a given sequence is unavoidable. Several commercial vendors currently provide sets of four synthetic shRNA, generally RNAi, sequences in which each sequence targets a different region of a gene to be silenced in order to increase the probability of reducing or eliminating gene expression of the targeted gene of interest. This strategy reduces the need to validate the effectiveness of individual shRNA, i.e., RNAi, sequences.

The U6 and H1 promoters have been used to express antisense RNA and more recently to express shRNA sequences that can silence gene expression. Gene silencing technology, either with synthetic RNAi or vector-based expression of shRNA has become an important tool for studying gene function, functional genomics, and drug target identification and validation. Gene silencing methods also hold great promise as potential therapies for treating diseases, including but not limited to AIDS, HBV and HCV infection, cancer, inflammatory diseases such as rheumatoid arthritis, and metabolic disorders. Five different human U6 genes have been identified. The present invention includes any human Pol III promoters, including those of the U6 and H1 type, that can be rearranged, reoriented or the like to yield functional bidirectional promoter that can direct transcription of two shRNA transcript at the same time. Likewise, because the U6 or H1 promoters are highly conserved in other species, control sequences derived from other species might also be substituted for those presented here to yield functional bidirectional promoters of the present invention. Thus, the invention also includes a basic strategy to yield numerous Pol III promoters that function as bidirectional promoters in gene silencing methods.

Generally, promoter embodiments of the present invention comprise 1) a complete Pol III promoter, which includes a TATA box, a Proximal Sequence Element (PSE), and a Distal Sequence Element (DSE); and 2) a second basic Pol III promoter that includes a PSE and TATA box fused to the 5′ terminus of said DSE in reverse orientation. The TATA box, which is named for its nucleotide sequence, is a major determinant of Pol III specificity. It is usually located at a position between nt. −23 and −30 relative to the transcribed sequence, and is a primary determinant of the beginning of the transcribed sequence. The PSE is usually located between nt. −45 and −66. The DSE enhances the activity of the basic Pol III promoter. In the U6 promoter, the DSE is usually located between nt. −190 and −260. In the H1 promoter, there is no gap between the PSE and the DSE. Two non-limiting examples of bidirectional promoters are presented. They are referred to as BiU6 and BiH1.

Turning now descriptively to the drawings, in which similar reference characters denote similar elements throughout the figures, which illustrate two examples of bidirectional Pol III promoters that can be used together, as well as independently, for the expression of multiple short RNA sequences. The resultant multiple shRNA sequences are generally different sequences, but can be the same sequence. The drawings represent only one class of Pol III promoters. This class, to which the U6 and H1 promoters belong, is distinguished from other classes of Pol III promoters by the location of its control elements, which are external to the transcribed sequence. The U6 and H1 promoters have been rendered bidirectional by implementing the features shown in FIG. 1. Each bidirectional promoter consists of 1) a complete, conventional, unidirectional Pol III promoter that contains 3 external control elements: a DSE, a PSE, and a TATA box; and 2) a second basic Pol III promoter that includes a PSE and a TATA box fused to the 5′ terminus of the DSE in reverse orientation. The TATA box, which is recognized by the TATA binding protein, is essential for recruiting Pol III to the promoter region. Binding of the TATA binding protein to the TATA box is stabilized by the interaction of SNAPc with the PSE. Together, these elements position Pol III correctly so that it can transcribe the expressed sequence. The DSE is also essential for full activity of the Pol III promoter (Murphy et al, 1992, Mol. Cell Biol. 12:3247-3261; Mittal et al., 1996, Mol. Cell Biol. 16:1955-1965; Ford and Hernandez, 1997, JBC, 272:16048-16055; Ford et al., 1998, Genes, Dev., 12:3528-3540; Hovde et al., 2002, Genes Dev. 16:2772-2777). Transcription is enhanced up to 100-fold by interaction of the transcription factors Oct-1 and/or SBF/Staf with their motifs within the DSE (Kunkel and Hixon, 1998, Nulc. Acid Res., 26:1536-1543). Since the forward and reverse oriented basic promoters direct transcription of sequences on opposing strands of the double-stranded DNA templates, the positive strand of the reverse oriented basic promoter is appended to the 5′ end of the negative strand of said DSE. Transcripts expressed under the control of either the U6 or the H1 promoter are terminated by an unbroken sequence of 4 or 5 T's. Since transcription in the forward and reverse orientations proceeds on opposite DNA strands, this string is depicted in FIG. 1 as 5 A's for the reverse oriented basic promoter.

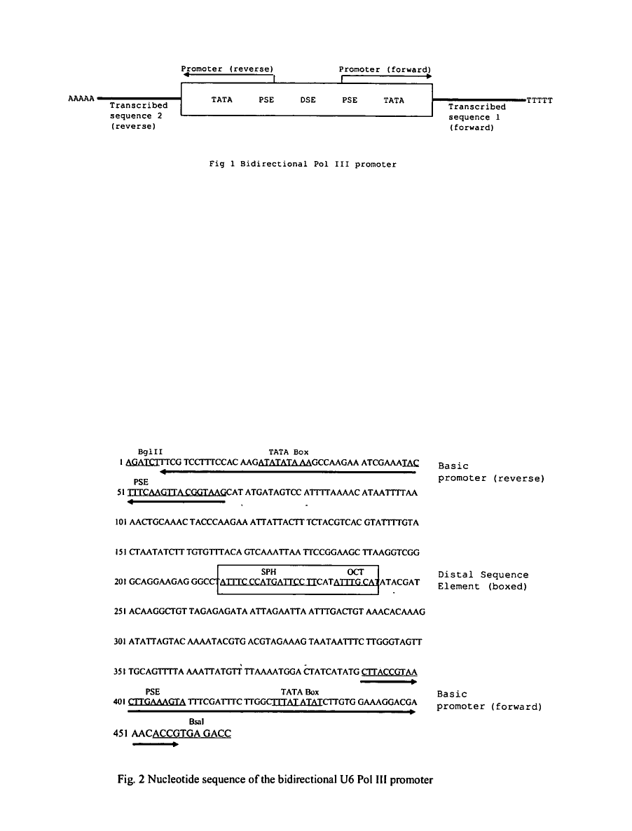

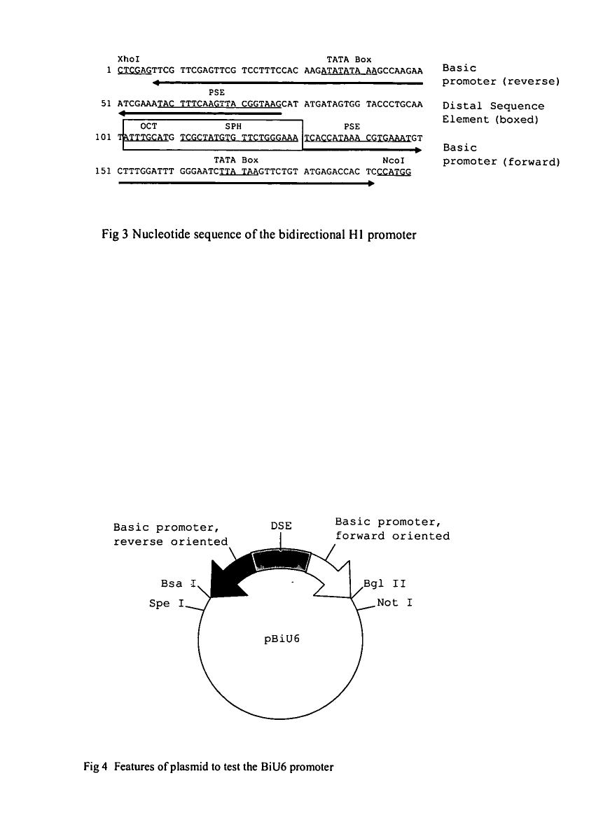

Although the U6 and H1 promoters both have the basic structure shown in FIG. 1, they differ in their PSE/DSE organization. The H1 promoter is more compact than the U6 promoter. In the U6 promoter, the DSE is separated from the adjoining PSE and TATA box by about 150 nucleotides, and this spacing is preserved when a second PSE and TATA box are appended in reverse orientation, as shown in FIG. 2. In the H1 promoter, the DSE is adjacent to the PSE and the TATA box (Myslinski et al., 2001, Nucl. Acid Res. 29:2502-2509). To minimize sequence repetition, this promoter was rendered bidirectional by creating a hybrid promoter, in which transcription in the reverse direction is controlled by appending a PSE and TATA box derived from the U6 promoter, as shown in FIG. 3. To facilitate construction of BiH1, a small spacer sequence was also inserted between the reverse oriented basic promoter and the DSE. In light of the satisfactory performance of this construct, it appears that spacing is not critical in Pol III promoter performance since spacing of 10-15 nucleotides and 150 nucleotides worked equally well.

Further, the bidirectional Pol III promoter of the present invention is used alone, in combination with one or more bidirectional Pol III promoters and/or in combination with one or more unidirectional Pol III promoters.

### EXAMPLE 1

### Construction of Bidirectional U6 Promoter

The PCR method was used to amplify human U6 promoter from the genomic DNA extracted from human 293 cells by using the following primers (forward primer: ′5-CGCAAGCTTAAGGTCGGGCAGGAAGAGGGCCTA-3′; reverse primer: 5′-CCACTAGTGGGTCTCACGGTGTTTCGTCCTTTCCAC-3. In these two primers, restriction site Hind III, Bsa I and Spe I (underlined) were designed to facilitate the cloning, respectively. PCR fragment was cut with Hind III and Spe I and ligated into the Hind III/Spe I sites of pLEP4CMV vector to obtain a plasmid pLEPU6.

In order to amplify the minimal U6 promoter that contains the TATA box and PSE, two PCR primers (forward primer: ′5-CGCAAGCTTCCGGAATTAATTTGACTGTAAACAC-3′; reverse primer: 5′-GGAATTCTAGCGGCCGCGAAGATCTTTCGTCCTTTCCACAAGATA-3′) were synthesized. In these two primers, restriction site Hind III, EcoR I, Not I and Bgl II were added, respectively. PCR amplified fragment was cut by Hind III and EcoR I and was ligated into the Hind III/EcoR I sites of pLEPU6 to obtain a plasmid pBiU6. The insert was verified by DNA sequencing and the results are shown in FIG. 2 (SEQ ID NO. 1). The structure of pBiU6 is illustrated in FIG. 4.

In summary, a bidirectional U6 promoter was obtained by inverted the minimal U6 promoter elements, TATA box and PSE, adjacent to the DSE element. It was anticipated that a single DSE should be able to enhance the two basic U6 promoter activity in opposite orientation. The final plasmid, pBiU6, contains restriction enzyme sites placed to facilitate the cloning of shRNA oligonucleotides behind the forward or the reverse oriented arms of the promoter.

### EXAMPLE 2

### Construction of Expression Vectors Containing shRNA Against Reporter Gene, β-galactosidase

To demonstrate that transcription proceeds in both directions, shRNA expression vectors were constructed. First pLEPU6lacZ was generated by inserting a pair of shRNA oligos against β-galactosidase gene (Lacz-sense: 5′-ACCGGCGTTTCATCTGTGGTGCTTCTAGAGAGCACCACAGATGAAACGCCCTTTTTG-3′; Lacz-antisense:5′-GATCCAAAAAGGGCGTTTCATCTGTGGTGCTCTCTAGAAGCACCACAGATGAAACG C-3′) into pLEPU6 vector predigested with Spe I and Bsa I. The resultant plamid was termed pU6lacZ. The same shRNA sequence also was used to construct two plasmids, pBiU6lacZ1 and pBiU6lacZ2. The oligos encoding the shRNA were synthesized, annealed and cloned into the forward direction of bidirectional U6 promoter at pBiU6 digested with Spe I and Bsa I to generate pBiU6lacZ1 (forward). Similarly, the synthetic oligonucleotides with appropriate restriction ends were made and inserted into Not I/Bgl II sites of pBiU6 to obtain the expression vector pBiU6lacZ2 (reverse).

### EXAMPLE 3

### Effective Silencing Expression of β-galactosidase Gene by pBiU6 Plasmids

The efficiency of the bidirectional promoters to direct expression of two RNAi was evaluated by measuring a reporter gene, β-galactosidase activities in human 293 cells or Mouse L cells. Unless otherwise emphasized in the description, all results are either a representative of three independent experiments or a summary of results from three experiments.

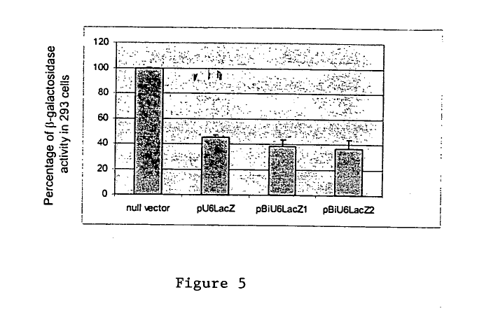

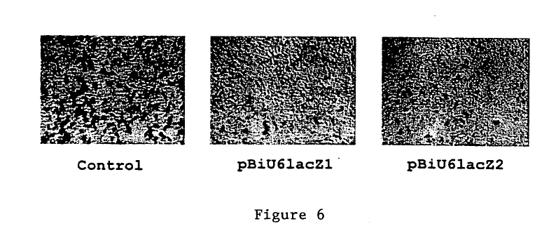

pU6lacZ, pBiU6lacZ1 and pBiU6lacZ2 plasmid DNA was purified using Qiagen Plasmid Purification Kit and diluted to 0.2 μg/μl with TE buffer. Before performing the transfecttion experiment, the human 293 cells were split into 6 well plates to reach 80% confluence. The transfection was carried out using Invitrogen's Lipofectamine 2000 according to manufacturer's instructions. Briefly, one ug of pU6lacZ, pBiU6lacZ1 or pBiU6lacZ2 DNA was cotransfected into 293 cells with 1 ug of expression vector, pLEPCMV-lacZ, which expressed β-galactosidase at high level in many cell lines. As a control, pLEPCMV-LacZ was also co-transfected with the null vector pU6 that does not contain any shRNA sequence. Fourty eight hours post transfection, β-galactosidase activity assay was performed using Promega's β-galactosidase enzyme assay system according to manufacturer's instructions. Briefly, after removing the growth media, the cells were washed twice with PBS. 400 ul lysis buffer was added into the wells and incubated at 37° C. for 15 min. After cell lysis, the cell lysate was transferred into a microcentrifuge tube and centrifuged for 5 min to spin down the cell debris. The β-galactosidase enzyme activity was assayed by adding 50 μl of lysate into 100 μl of reaction buffer. After incubation at 37° C. for 15 min, the reaction was stopped and measured the absorbance at 420 nm on a spectrophotometer (SHIMADZU UV-1601). The experiment results showed the expression of β-galactosidase was significantly suppressed in cells transfected by pU6lacZ, pBiU6lacZ1 and pBiU6lacZ2 plasmids (FIG. 5). The suppression is presumably due to the RNAi processed from expressed shRNA. More importantly, the levels of suppression were comparable in pU6lacZ, pBiU6lacZ1 and pBiU6lacZ2 transfected cells. These results suggested that the transcription activity of each direction from the BiU6 promoter is comparable to that of the conventional U6 promoter and incorporation of a second set of minimal U6 promoter at the reverse orientation to the DSE does not interfere with either the DSE or the natural basic promoter function. The reduction of gene expression by pBiU6lacZ1 and pBiU6lacZ2 was also measured by staining β-galactosidase activity in 293 cells. The plasmid, pBiU6lacZ1 or pBiU6lacZ2 were co-transfected into 293 cells with a β-galactosidase expression vector as described above. After 48 hours, the cells were stained with Mirus's β-galactosidase staining kit according to manufacturer's instructions. The results showed both plasmids greatly reduced expression of the reporter gene, β-galactosidase, demonstrating that transcription proceeds at effective rates from both the forward and reverse oriented elements of BiU6 (FIG. 6).

### EXAMPLE 4

### Construction of Bidirectional H1 Promoter

Similar strategy was employed to generate bidirectional H1 promoter as did for the BiU6 promoter described in Example 1. First, the human H1 promoter was amplified by PCR from the genomic DNA extracted from human 293 cells by using the following primers (forward primer: ′5-CGCGGATCCTTAATTAAGGTACCCTGCAATATTTGCATGTCGCTA-3′; reverse primer: 5′-CTATCGATAGATGCCATGGGAGTGGTCTCATACAGAAC-3′). In these two primers, restriction site BamH I, Kpn I, Cla I and Nco I were added, respectively. PCR amplified H1 fragment was cut by BamH I and Cla I and ligated into the BamH I/Cla I sites of pBiU6 vector to obtain a plasmid pBiU6H1.

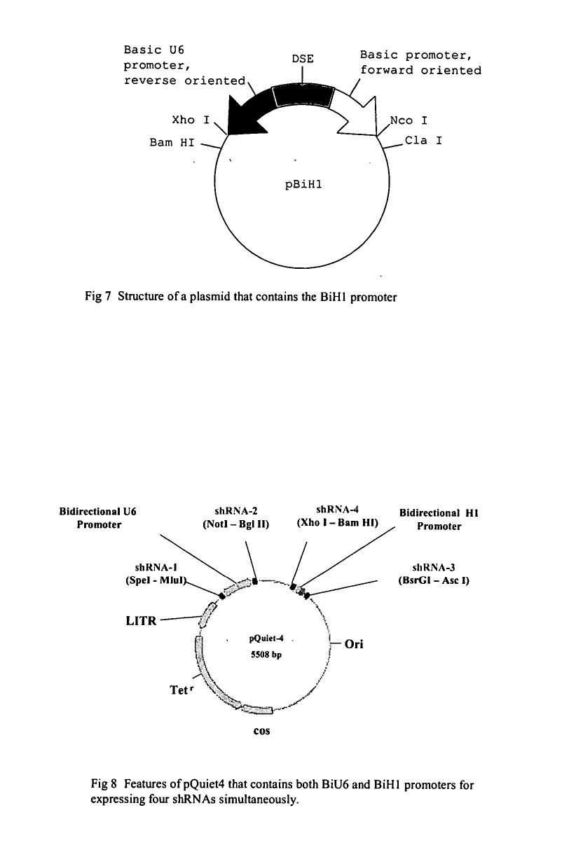

The next step was to make the H1 promoter bidirectional. However, the H1 promoter is more compact than the U6 promoter. In the H1 promoter, the DSE is adjacent to the PSE and the TATA box (Myslinski et al., 2001, Nucl. Acid Res. 29:2502-2509). To minimize sequence repetition in such a close location, this promoter was rendered bidirectional by creating a hybrid promoter by appending a PSE and TATA box derived from the U6 promoter. Two PCR primers were designed for inserting the basic U6 elements into H1 promoter but in reversed orientation relative to the H1 natural PSE. The PCR primers sequences are: forward primer: ′5-CGCGGTACCACTATCATATGCTTACCGTAAC-3′; reverse primer: 5′-GCGGATCCTCGCCCGGGACTCGAGTTCGTCCTTTCCACAAGATA-3′. In these two primers, restriction site Kpn I, BamH I and Xho I were added, respectively. PCR amplified fragment was cut by Kpn I and BamH I and ligated to Kpn I/BamH I digested pBiU6H1 to obtain a plasmid pBiU6BiH1. The organization of this H1/U6 hybrid promoter is diagramed in FIG. 7 and its nucleotide sequence shown in FIG. 3 (SEQ ID NO: 2). Therefore the pBiU6BiH1 plasmid contains two bidirectional promoters and should be able to express four shRNA simultaneously. The DNA sequences were confirmed by sequencing.

### EXAMPLE 5

### Insertion of Stop Sequence into pBiU6BiH1 Plasmid

Previous reports showed that five consecutive T residues are sufficient to terminate the transcription activity of Pol III promoter (Booth, B. L., Jr. 1997, J. Biol. Chem. 272: 984-991 and Tazi, J. et al. 1993, Mol. Cell. Biol. 13:1641-1650). To prevent any undesired transcription from the unoccupied positions of bidirectional U6 and H1 promoter, four pairs of stop sequences were designed and synthesized: 1) Stop1-top: 5′-ACCCGGTTTTTGAATTCA-3′ and Stop1-bot: 5′-CTAGTGAATTCAAAAAC-3′, were cloned into the forward direction of BiU6 promoter; 2) Stop2-top: 5′-GATCTGTTTTTGAATTCGC-3′ and Stop2-bot: 5′-GGCCGCGAATTCAAAAACA-3′ were cloned into the reverse direction of BiU6 promoter; 3. Stop3-top: 5′-CATGGTTTTTGAATTCAT-3′ and Stop3-bot: 5′-CGATGAATTCAAAAAC-3′ were cloned into the forward direction of BiH1 promoter; 4. Stop4-top: 5′-TCGAGTTTTTGAATTCG-3′ and Stop4-bot: 5′-GATCCGAATTCAAAAAC-3′ were cloned into the reverse direction of BiH1 promoter, respectively. The resultant vector was termed pQuiet-4 for its capability to express four shRNAs simultaneously. The vector structure was confirmed by complete sequencing (SEQ ID NO: 3) and a map is shown in FIG. 8.

### EXAMPLE 6

### Construction of pQuiet Vectors Expressing the Same RNAi Sequence at Each of 4 Positions in pQuiet-4 Vector

To evaluate the performance of the BiU6 and BiH1 promoters in directing the expression of shRNA, four shRNA sequences that result in the identical RNAi expression were designed and constructed, one at a time into, each of the four positions in pQuiet-4 vector. The other 3 positions remain unoccupied and contain only the stop sequence as described above. The first plasmid, pQuiet4lacZ1, was constructed by inserting a pair of RNAi oligos designed to silence β-galactosidase gene (LacZ9-top: 5′-CGCGGAAGTGACCAGCGAATACCTTCTAGAGAGGTATTCGCTGGTCACTTCTTTTTA-3′; LacZ9-bot: 5′-CTAGTAAAAAGAAGTGACCAGCGAATACCTCTCTAGAAGGTATTCGCTGGTCACTTC-3′) into the forward direction of BiU6 promoter in pQuiet-4 digested with Spe I and Mlu I (also refer to position 1). Same strategy was used to generate other three pQuiet4 vectors using oligonucleotides with ends altered accordingly for cloning into each of the three positions (see pQuiet4 map in FIG. 8 for restriction sites). The resultant vectors are termed pQuiet4lacZ2 (reverse direction of BiU6, position 2), pQuiet4lacZ3 (forward direction of BiH1, position 3) and pQuiet4lacZ4 (reverse direction of BiH1, position 4), respectively.

### EXAMPLE 7

### Effective Suppression of β-galactosidase Gene Expression by a Single Expressed RNAi at Each Position of pQuiet-4 Vectors

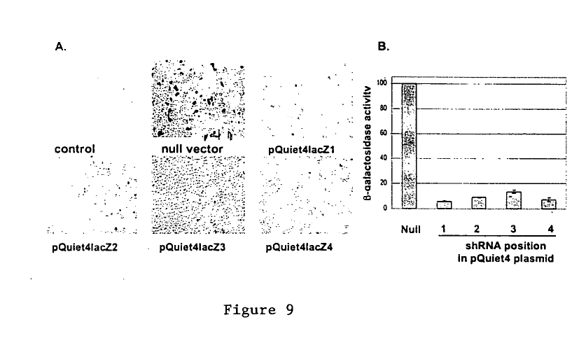

The four plasmids, pQuietlacZ1, pQuietlacZ2, pQuietlacZ3 and pQuietlacZ4 were each co-transfected along with a β-galactosidase expression vector, pLEPCMV-lacZ into mouse L cells. The null pQuiet4 vector was also co-transfected with pLEPCMV-lacZ into mouse L cells as negative control. Two days post transfection, the expression of β-galactosidase was evaluated quantitatively by using a using Promega's β-galactosidase enzyme assay system to assess β-galactosidase activity. The results indicated that all 4 positions in the pQuiet-4 vector were capable of expressing the desired shRNA sequence at a level sufficient to suppress β-galactosidase more than 85% and the highest suppression levels reached >95% (FIG. 9B). More importantly, it appeared that the same RNAi sequence expressed from all four positions showed comparable levels of inhibition. These results strongly demonstrated that both forward and reverse orientation of BiU6 and BiH1 promoters are active in directing the expression of shRNA sequences.

The β-galactosidase activity staining was also performed to evaluate the suppression by expressed RNAi from the pQuiet4lacZ1, pQuiet4lacZ2, pQuiet4lacZ3 and pQuiet4lacZ4 vectors. All four plasmids were able to reduce expression of the reporter gene β-galactosidase, as shown in FIG. 9A.

### EXAMPLE 8

### Construction of a pQuiet Vector that Expresses shRNAs to Target Three Reporter Genes Simultaneously

To test the potential of the pQuiet-4 vector to silence the expression of multiple genes, three RNAi sequences targeting β-galactosidase, firefly luciferase, and green fluorescent protein (GFP) genes were designed and constructed into pQuiet-4 vector. The shRNA sequences are: lacZRNAi: LacZ9-top: 5′-CGCGGAAGTGACCAGCGAATACCTTCTAGAGAGGTATTCGCTGGTCACTTCTTTTTA-3′ and LacZ9-bot: 5′-CTAGTAAAAAGAAGTGACCAGCGAATACCTCTCTAGAAGGTATTCGCTGGTCACTTC-3′; GFPRNAi: GFP-top: 5′-GATCGAAGCAGCACGACTTCTTCTTCTAGAGAGAAGAAGTCGTGCTCTTCTTTTTGC-3′ and GFP-bot: 5′-GGCCGCAAAAAGAAGCAGCACGACTTCTTCTCTCTAGAAGAAGAAGTCGTGCTGCT TCG-3′; LucRNAi: Luc-top: 5′-GTACGCTGAGTACTTCGAAATGTCTTCTAGAGAGACATTTCGAAGTACTCAGCTTTT TGG-3′; and Luc-bot: 5′-CGCGCCAAAAAGCTGAGTACTTCGAAATGTCTCTCTAGAAGACATTTCGAAGTACTC AGC-3′. The LacZRNAi, GFPRNAi and LucRNAi was inserted into position 1 (forward BiU6), 2 (reverse BiU6) and 3 (forward BiH1), respectively in pQuiet-4. The resultant vector was termed as pQuietlacZgfpluc.

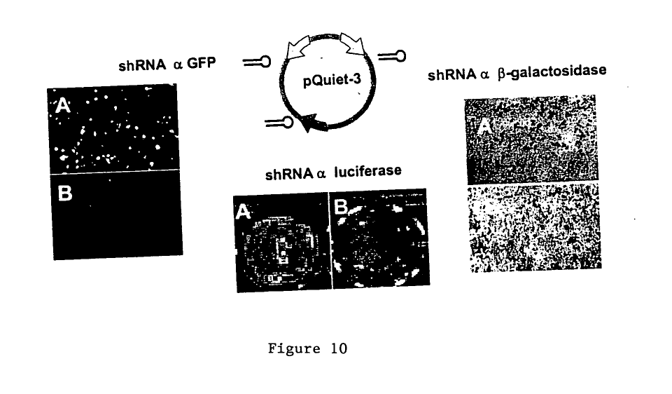

Again transient transfection in was carried out in mouse L cells for suppression of the reporter genes. Briefly, mouse L cells were seeded in a six well plate a day prior to the transfection experiment to reach confluence about 80%. Three reporter plasmids, pLEPCMV-LacZ, pLEPCMVGFP and pGL3Luc (Progema) were employed in the silencing experiments. The transfection was setup as followings: one μg of the pQuietlacZgfpluc or 1 μg of the pQuiet-4 null vector was mixed with 0.5 μg of pLEPCMV-LacZ, 0.5 μg of pLEPCMVGFP and 0.5 μg of pGL3Luc. The DNA mixture were diluted with Opti-MEM medium (Invitrogen) and mixed with Lipofectamin 2000 transfection reagent (Invitrogen) as described above and applied onto mouse L cells. Fourty eight hours post tranfection, the expression of the three reporter genes was analyzed sequentially from the same wells. First, GFP expression was assessed by fluorescent microscopy and recorded by photography; Secondly, luciferase activity was examined by adding 10 μl of luciferease substrated and recorded on Xenogen IVIS imaging station; and finally, mouse L cells were fixed and stained with X-gal solution to detect the β-galactosidase expression following standard protocol. Results are shown as FIG. 10. In summary, the expression of all three reporter genes was suppressed to almost background levels by a single plasmid, pQuietlacZgfpluc. In contrast, no suppression was detected in the null vector transfected cells. These results demonstrated in principle that multiple gene silencing is achieved by using single vector (i.e. pQuietlacZgfpluc) that expresses multiple shRNA sequences.

### EXAMPLE 9

### Construction of a Vector to Simultaneously Target p38 Mitogen-Activated Protein Kinase (MAPK) Isoforms

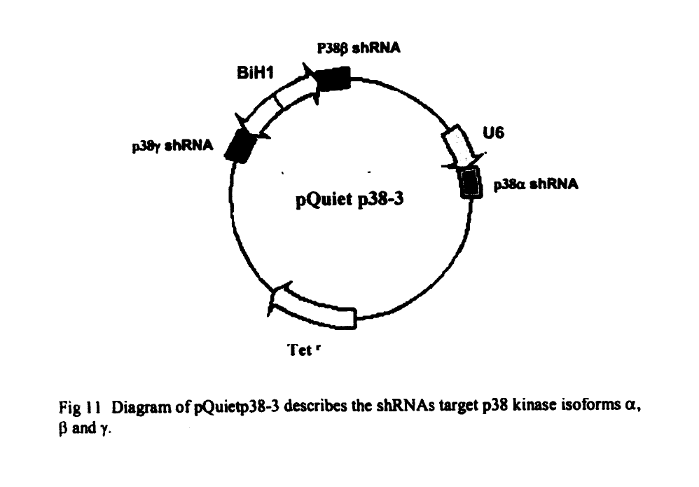

As described above, the pQuietlacZgfpgl3 carrying three RNAi successfully inhibited the expression of three reporter genes (FIG. 10). In order to test if the endogenous genes can be silenced by shRNAs expressed from pQuiet4 vector, a pQuiet vector carrying three RNAi against three p38 MAPK isoforms was constructed. The p38 MAPK are a family of protein kinases that play important roles in cellular responses to external stress signals. So far four mouse p38 isoforms (p38α, p38β, p38δ and p38γ) have been reported (Kumar, S. et al. 2003, Nat Rev Drug Discov. 2:717-26). We designed 3 RNAi targeting p38α, p38β, and p38γ isoforms: 1) p38αRNAi: sense strand: 5′-CGCGGCTGCTGCCGCTGGAAGATTTCTAGAGAATCTTCCAGCGGCAGCAGCTTTTTA-3′ and antisense-strand: 5′-CTAGTAAAAAGCTGCTGCCGCTGGAAGATTCTCTAGAAATCTTCCAGCGGCAGCAGC-3′; 2) p38βPRNAi: sense-strand: 5′-GTACGGCAAGAGCTGAACAAAACTTCTAGAGAGTTTTGTTCAGCTCTTGCCTTTTTG G-3′ and antisense-strand: 5′-CGCGCCAAAAAGGCAAGAGCTGAACAAAACTCTCTAGAAGTTTTGTTCAGCTCTTGC C-3′; 3. p38γRNAi: sense-strand: 5′-TCGAGTGTACCAAGACCTGCAGCTTCTAGAGAGCTGCAGGTCTTGGTACACTTTTTG-3′ and antisense-strand: 5′-GATCCAAAAAGTGTACCAAGACCTGCAGCTCTCTAGAAGCTGCAGGTCTTGGTACAC-3′. The p38αshRNA, p38γshRNA and p38βshRNA were respectively inserted into the position 1 (BiU6 forward position), position 3 (BiH1 forward position), position 4 (BiH1 reverse position) and in pQuiet-4 respectively to generate plasmid pQuietp38-3. The plasmid map is illustrated in FIG. 11.

### EXAMPLE 10

### Conversion of pQuiet38-3 into Adenovirus Vector

One major obstacle in achieving high efficiency gene silencing is delivery of RNAi into target cells or hosts. All above examples employed a co-transfection of pQuiet plasmids with their target gene expression vectors. Thus the cells were transfected by the reporter gene(s) likely would also got transfected by the pQuiet vector DNA. In order to suppress any endogenous gene in host cells, a near 100% delivery efficiency is needed to achieve maximal gene silencing. To achieve this high efficient RNAi delivery, a recombinant adenoviral vector was constructed to contain the pQuiet4p38 elements to test the silencing of endogenous p38 kinase isoforms in mouse L cells. Adenovirus infection is highly dependent on the expression of its CAR (coaxakie adenovirus receptor) receptor on the host cell surface. A mouse L cell line that stably expresses high levels of CAR receptor was established and this cell line is termed MLCAR (Du et al, 2004, unpublished data). The optimal multiplicity of infection (moi) was first determined using an AdCMV-β-gal vector. Moi of 1,000-10,000 (viral particles/cell) were determined to be the range of maximal gene expression with minimal vector toxicity. Although adenovirus infection itself up-regulates the expression of endogenous p38 kinases, it has been shown that this is a transient induction and should be resolved 48 hours after infection (Bhat, N. R. and Fan, F. 2002, Brain Res. 948:93-101).

The pQuiet38-3 was inserted into an adenovirus according to a method described by Wang, X. et al.(2000, J. Virology 74:11296-303). Briefly, the pQuiet38-3 was digested with Cla I and ligated to a linearized pREP plasmid that contains the remaining adenovirus genome. The ligation product is packaged into a cosmid using Epicentre's lambda phage packaging kit. The packaging products were used to infect E. coli cells. After incubation overnight, the positive clones were selected, and cosmid DNA were purified. The purified cosmid DNA (2 μg) was digested with I-Ceu I and then transfected into 293 cells with Lipofectamine 2000 according to manufacturer's instructions. The 293 cells were grown at 37° C. with 5% CO₂. The adenovirus plaques were seen 8 days after transfection. A low titer of virus (approximately 10⁹ virus particles (vp)/ml) can be obtained from a single well at 6-well plate. The low titer of virus was further amplified to 10¹² vp/ml. The amplified adenoviruses were purified, and used to infect MLCAR cells. This adenoviral vector is termed AdQuietp38-3.

### EXAMPLE 11

### Silencing Expression of p38α, p38β and p38γ Genes Using AdQuietp38-3

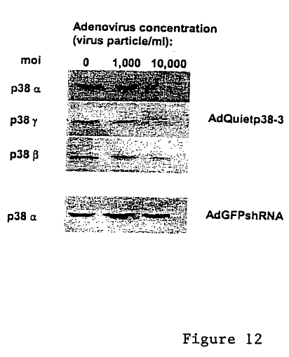

To test the silencing of endogenous p38 kinases, MLCAR cells were seeded in six well plates a day prior to experiment to reach 80% confluence. MLCAR cells were infected by the AdQuietp38-3 vector or a control AdRNAi vector, AdGFPshRNA, at moi of 0, 1,000 and 10,000. Cells were incubated in a CO₂ incubator for addition two days. At day three post infection, cells were lysed by adding 250 μl of SDS-PAGE sample buffer and harvested for Western blot analyses. 25 μl of the cell lysates were resolved in a 10% SDS-PAGE gel and transferred onto a PVDF membrane following standard procedure. The membranes were probed with antisera specific to mouse p38α (BD Clontech), p38β ( Santa Cluz) or p38γ (R&D System). Western blot results are shown in FIG. 12. Results demonstrated suppression of all three p38 isoforms: p38α>80%, p38γ˜90% and p39β˜50% (FIG. 12, upper panels). In contrast no p38a inhibition was observed in the cells infected with AdGFPshRNA vector at all moi (FIG. 12, lower panel). The relatively lower silencing efficiency of p39β may be resulted from the sub-optimal RNAi sequence selection. These results clearly demonstrated the great potential (in combination with adenoviral vector) of the pQuiet4, or a similar vector, in silencing multiple endogenous genes simultaneously.

Thus, in combination with other viral vectors, such as lentivirus and adeno-associated virus vectors, or the like, long-term, gene suppression can be achieved in cultured cells as well as in living hosts. Further, this technology based on the many utilities of bidirectional promoters should provide a powerful tool for functional genomic studies, to dissect redundant signal transduction pathways and to be applied in treatment of disease states by targeting genes related to the disease phenotype.

Further, multiple RNAi expression systems as disclosed herein should allow reduced validation effort in selecting an effective RNAi sequence. More than one shRNA sequences can be inserted into the pQuiet-4 vector or a like vector to target the same gene. Individually or in combination, these shRNA molecules likely will increase the chance and efficiency to silence the target one or more genes without RNAi sequence validation.

The above description is sufficient for one skilled in the art to determine the optimum relationships for the parts of the invention, to include variations in size and nucleotide sequence of the elements and intervening sequences without undue experimentation, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

### REFERENCES

Bhat N R, Fan F. 2002. Adenovirus infection induces microglial activation: involvement of mitogen-activated protein kinase pathways, Brain Research, (948): 93-101.
Booth B L Jr, Pugh B F. 1997. Identification and characterization of a nuclease specific for the 3′ end of the U6 small nuclear RNA, J Biol Chem. 272(2):984-91.
De Wilde C, Van Houdt H, De Buck S, Angenon G, De Jaeger G, Depicker A. 2000. Plants as bioreactors for protein production: avoiding the problem of transgene silencing. Plant Mol. Biol. 43:347-359.
Ford E, Strubin M, Hernandez N. 1998. The Oct-1 POU domain activates snRNA gene transcription by contacting a region in the SNAPc largest subunit that bears sequence similarities to the Oct-1 coactivator OBF-1, Genes Dev. 12(22):3528-40.
Ford E, Hernandez N. 1997, Characterization of a trimeric complex containing Oct-1, SNAPc, and DNA. J Biol Chem. 272(25):16048-55
Hovde S, Hinkley C S, Strong K, Brooks A, Gu L, Henry R W, Geiger J. 2002. Activator recruitment by the general transcription machinery: X-ray structural analysis of the Oct-1 POU domain/human U1 octamer/SNAP190 peptide ternary complex, Genes Dev. 16(21):2772-7.
Kumar S, Boehm J, Lee J C. 2003. p38 MAP kinases: key signalling molecules as therapeutic targets for inflammatory diseases, Nat Rev Drug Discov. (9):717-26. Review.
Kunkel G R, Hixson J D. 1998.The distal elements, OCT and SPH, stimulate the formation of preinitiation complexes on a human U6 sh RNA gene promoter in vitro, Nucleic Acids Res. 26(6):1536-43.
Mittal V, Cleary M A, Herr W, Hernandez N. 1996. The Oct-1 POU-specific domain can stimulate small nuclear RNA gene transcription by stabilizing the basal transcription complex SNAPc, Mol Cell Biol. 16(5):1955-65.
Murphy S, Yoon J B, Gerster T, Roeder R G. 1992. Oct-1 and Oct-2 potentiate functional interactions of a transcription factor with the proximal sequence element of small nuclear RNA genes, Mol Cell Biol. 12(7):3247-61.
Myslinski E, Ame J C, Krol A, Carbon P. 2001. An unusually compact external promoter for RNA polymerase III transcription of the human H1RNA gene, Nucleic Acids Res. 29(12):2502-9.
Tazi J, Forne T, Jeanteur P, Cathala G, Brunel C. 1993. Mammalian U6 small nuclear RNA undergoes 3′ end modifications within the spliceosome, Mol Cell Biol. 13(3):1641-50.
Tuschl, T. 2002. Expanding small RNA interference, Nat Biotechnol. 20(5):446-8. No abstract available.
Wang X, Zeng W, Murakawa M, Freeman M W, Seed B. 2000. Episomal segregation of the adenovirus enhancer sequence by conditional genome rearrangement abrogates late viral gene expression, J Virol. (23):11296-303.

1. A bidirectional promoter, comprising:
control elements that provide for transcription of short RNA sequences in one direction; and control elements added in reverse orientation that provide for transcription of short RNA sequences in the opposite direction.

2. The RNA polymerase III bidirectional promoter as shown in SEQ ID NO: 1.

3. The RNA polymerase III bidirectional promoter as shown in SEQ ID NO: 2.

4. A plasmid comprising:
elements that facilitate propagation in a bacterial host and subsequent propagation in eukaryotic cells; and
one or more bidirectional RNA polymerase III promoters.

5. An adenovirus comprising:
elements that facilitate propagation of the virus in eukaryotic cells; and
one or more bidirectional promoters.

6. A vector for directing expression of two or more different short RNA sequences comprising one or more bidirectional RNA polymerase III promoters to direct transcription of the short RNA sequences.

7. The vector of claim 5 further comprising termination elements for termination of transcription of the short RNA sequences.

8. The vector of claim 6 wherein the vector is selected from the group consisting of a plasmid, an adenovirus, an adeno-associated virus, a retrovirus and a lentivirus.

9. The method of claim 8 further comprising a unidirectional promoter.

10. The method of claim 9 wherein the number of short RNA sequences expressed is 2, 3 , 4 or 5.

11. A method of silencing the expression of one or more genes comprising the expression of two or more short RNA sequences under the direction of one or more bidirectional RNA polymerase III promoters from a suitable vector.

12. The method of claim 11 wherein the vector is selected from the group consisting of a plasmid, an adenovirus an adeno-associated virus and a lentivirus.

13. A method for increasing the number of short RNA sequences that are expressed under the direction of elements of a single vector comprising the inclusion of one or more bidirectional RNA polymerase III promoters in the vector.

14. The method of claim 13 wherein the vector is selected from the group consisting of plasmid, an adenovirus an adeno-associated virus and a lentivirus.

15. A method for treating a disease state in a subject comprising administration of a vector that includes one or more bidirectional RNA polymerase III promoters.

16. The vector of claim 15, wherein the vector is selected from the group consisting of a plasmid, an adenovirus, an adeno-associated virus, a retrovirus and a lentivirus.

17. A method for treating an infectious disease in a subject comprising targeting two or more regions of a viral genome, wherein targeting the two or more regions of the viral genome comprises administering a vector to the subject that includes one or more bidirectional RNA polymerase III promoters that direct the expression of two or more viral-related short RNA sequences to yield suppression of the expression of the viral genome.

18. The method of claim 17, wherein the vector is selected from the group consisting of a plasmid, an adenovirus, an adeno-associated virus, a retrovirus and a lentivirus.

19. A method for minimizing the size of control elements that direct transcription of short RNA sequences comprising the inclusion of one or more bidirectional RNA polymerase III promoters in said control elements.

20. A kit comprising one or more bidirectional promoters; and
instructions for use.

21. The kit of claim 20 further comprising one or more accessory elements for the construction of a vector that provide for the expression of two or more short RNA sequences of interest under the direction of the one or more bidirectional promoters.

22. The kit of claim 20 wherein the vector is selected from the group consisting of a plasmid, an adenovirus, an adeno-associated virus, a retrovirus and a lentivirus.

23. A kit comprising a vector comprising one or more bidirectional promoters, wherein the vector comprises the means for insertion of DNA sequences corresponding to two or more short RNA sequences of interest under the direction of the one or more bidirectional promoters; and
instructions for use.

24. The kit of claim 23 wherein the vector comprises suitable termination elements to yield the desired short RNA sequences of interest.

25. The kit of claim 23 wherein the vector is selected from the group consisting of a plasmid, an adenovirus, an adeno-associated virus, a retrovirus and a lentivirus.
